(19) United States
(12) Patent Application Publication
(10) Pub. No.: US 2011/0160617 A9
(43) Pub. Date: Jun. 30, 2011

(54) CLINICAL ASSESSMENT SCALES AND METHODS

(75) Inventors: Jane A. Thomas, Oxnard, CA (US); Patricia S. Walker, Santa Barbara, CA (US)

(73) Assignee: ALLERGAN, INC., Irvine, CA (US)

(21) Appl. No.: 12/604,084

(22) Filed: Oct. 22, 2009

Related U.S. Application Data
(60) Provisional application No. 61/107,455, filed on Oct. 22, 2008.

(51) Int. Cl. A61B 5/107

(52) U.S. Cl. 600/590

(57) ABSTRACT

Disclosed herein are scales for assessing lip fullness, severity of perioral lines at rest, severity of perioral lines at maximum contraction or oral commissures. The scales comprise rows or columns of photographs corresponding to different severity categories. Each scale may have a Kappa score of at least about 0.700 for intra-rater and inter-rater reliability.

### RELATED APPLICATION

This application claims the benefit of and priority to U.S. Provisional Patent Application No. 61/107,455, filed on Oct. 22, 2008, the entire disclosure of which is incorporated herein by this specific reference.

### FIELD OF THE INVENTION

The present invention provides scales and methods for performing clinical assessment of an individual. Particularly, the present invention provides reliable scales to effectively rate lip fullness and/or the severity of perioral lines or oral commissures.

### BACKGROUND OF THE INVENTION

Expression lines and wrinkles on the face are caused by a number of factors including photodamage, smoking and muscular activity such as squinting and smiling. Expression lines particularly are characterized in part by the presence of grooves and lines in the area around the mouth, called perioral lines and oral commissures (lines which go down on either side of a person's mouth).

A common effect of the aging process is the loss of full and well-defined lips. Lip volume decreases, the visible red vermilion of the upper lip decreases and the central defining points (Cupid's bow) become blunted. The aging lip often develops perioral rhytides, which radiate outward from the vermilion border, as well as deepening oral commissures, which create a down-turn at the mouth corners. Genetic factors also contribute to lip fullness and contour as well as perioral wrinkling. Over the years, various substances have been injected into the lips for cosmetic enhancement.

There are a variety of materials and methods used for lip augmentation. Some of the current techniques provide for temporary lip augmentation by means of injection of various materials into the lip such as fat, collagen, hyaluronic acid, and particulated dermis or fascia. Other techniques, such as liquid silicone injections, provide a more permanent lip augmentation. Another permanent lip augmentation technique is the implantation of expanded polytetrafluoroethylene (PTFE) such as Gore-Tex® strips.

Based on the development of treatments for perioral lines, oral commissures and lip augmentation procedures, there is a need for reliable scales to effectively and consistently rate lip fullness and the severity of perioral lines or oral commissures. Such scales would be useful in both clinical practice and clinical trial research. Particularly, in clinical trial research, objective quantification is critical to measure the efficacy of an investigational treatment by comparing the severity of a condition before treatment to that measured after treatment. For a new treatment to achieve regulatory approval for marketing, its efficacy must be documented in clinical trials. Valid and reliable outcome measures are also important in evidence-based medicine to provide comparisons among similarly designed trials in the literature.

Accordingly, there is a need for reliable and consistent devices and methods useful for assessing, quantifying or rating lip conditions, for example, lip fullness and/or severity of perioral lines and/or oral commissures.

### SUMMARY OF THE INVENTION

Provided herein are scales, systems of scales, and methods for performing clinical assessment of an individual. In one embodiment, scales and methods are provided for performing clinical assessment of an individual that includes determining a base clinical assessment for the patient by generating information on a clinical rating scale. Particularly provided are scales and methods utilizing reliable and consistent scales to effectively rate lip fullness and/or the severity of perioral lines or oral commissures.

In one embodiment, a scale is provided for assessing at least one characteristic of an individual wherein the characteristic is lip fullness, severity of perioral lines at rest, severity of perioral lines at maximum contraction or oral commissures. The scale comprises rows or columns of photographs corresponding to different severity categories. In some embodiments, the scale has a Kappa score of at least about 0.700 for intra-rater and inter-rater reliability.

In some embodiments, the scale includes word-based descriptions accompanying the rows or columns of photographs corresponding to different severity categories of the scale.

In another embodiment, the word-based descriptions include at least two of the descriptions consisting of the words “none”, “minimal”, “mild”, “moderate”, “marked” and “severe.”

In another embodiment, the word-based descriptions are followed by at least two explanatory words describing a feature commonly found in the row or column of photographs indicating the severity category.

In another embodiment, the photographs corresponding to different severity categories of the scale are presented in rows.

In another embodiment, each row includes three photographs.

In another embodiment, the scale includes four rows of photographs.

In yet another embodiment, at least one photograph on the scale comprises a marking to guide a rater's attention to an area of the photograph.

Another embodiment includes a scale for assessing at least one characteristic of an individual wherein the characteristic is lip fullness, severity of perioral lines at rest, severity of perioral lines at maximum contraction or oral commissures and wherein the scale comprises rows or columns of photographs corresponding to different severity categories of the scale and wherein the scale has a Kappa score of at least about 0.700 for intra-rater and inter-rater reliability, the scale includes word-based descriptions accompanying the rows or columns of photographs corresponding to different severity categories of the scale and wherein the word-based descriptions are followed by at least two explanatory words describing a feature commonly found in the row or column of photographs indicating the severity category.

Also disclosed herein are methods. In one embodiment the method facilitates the assessment of at least one characteristic of an individual wherein the characteristic is lip fullness, severity of perioral lines at rest, severity of perioral lines at maximum contraction or oral commissures and wherein method comprises: providing a scale comprising rows or columns of photographs corresponding to different severity categories of the scale wherein the scale has a score of at least about 0.700 for intra-rater and inter-rater reliability.

In another embodiment, the step of providing comprises providing the scale depicted in FIG. 1, the scale depicted in FIG. 2, the scale depicted in FIG. 3 and/or the scale depicted in FIG. 4.

In another embodiment, the step of providing comprises providing word-based descriptions accompanying the rows or columns of photographs corresponding to different severity categories on the scale.

In another embodiment, the step of providing comprises providing word-based descriptions, for example, “none”, “minimal”, “mild”, “moderate”, “marked” and/or “severe.”

In another embodiment, the step of providing comprises providing word-based descriptions followed by at least two explanatory words describing a feature commonly found in the row or column of photographs indicating the severity category.

In another embodiment, the step of providing comprises providing photographs corresponding to different severity categories of the scale in rows.

In another embodiment, the step of providing comprises providing three photographs in each row.

In another embodiment, the step of providing comprises providing four rows of photographs.

In yet another embodiment, the step of providing comprises providing a marking on at least one photograph wherein the marking guides a rater's attention to an area of the photograph.

Another embodiment includes a method for assessing at least one characteristic of an individual wherein the characteristic is lip fullness, severity of perioral lines at rest, severity of perioral lines at maximum contraction or oral commissures and wherein the method comprises providing a scale comprising rows or columns of photographs corresponding to different severity categories of the scale and wherein the scale has a Kappa score of at least 0.700 for intra-rater and inter-rater reliability, the scale includes word-based descriptions accompanying the rows or columns of photographs corresponding to different severity categories of the scale and wherein the word-based descriptions are followed by at least two explanatory words describing a feature commonly found in the row or column of photographs indicating the severity category.

### BRIEF DESCRIPTION OF THE DRAWINGS

FIG. 1 depicts a Lip Fullness (LFS) scale.

FIG. 2 depicts a Severity of Perioral Lines at Rest (POL) scale.

FIG. 3 depicts a Severity of Perioral Lines at Maximum Contraction (POLM) scale.

FIG. 4 depicts an Oral Commissures (OCS) scale.

### DETAILED DESCRIPTION

There is a need for reliable and consistent scales to effectively rate lip fullness and the severity of perioral lines or oral commissures. Such scales are important in clinical practice, and are sometimes important or even essential for clinical trial research. Particularly, in clinical trial research, objective quantification is critical to measure the efficacy of an investigational treatment by comparing the severity of the condition before treatment to that measured after treatment.

The present disclosure provides four lip-specific photographic scales. In certain embodiments each scale has four grades or points for the classification of aesthetic features of the lips and mouth area. The scales have high inter- and intra-rater agreement with their use.

Features evaluated in the scales can include (1) Lip Fullness (LFS); (2) Severity of Perioral Lines at Rest (POL); (3) Severity of Perioral Lines at Maximum Contraction (POLM); and (4) Oral Commissures (OCS). Each grade of each four-point scale can be depicted with three frontal-view photographs as examples of subjects that would fall within the particular grade. Additionally, in certain embodiments, the LFS scale can include lateral photographs, which can be important for assessing anterior projection as a function of fullness. In other certain embodiments, the area of interest can be outlined, shaded or otherwise marked on the photos. The range of grades on each scale can be intended to represent the full spectrum (minimum to maximum) for the specific assessed feature, and the photos can generally be arranged from left to right within each grade to represent the range from minimum to maximum. The photographs can be accompanied by brief written descriptions.

In one aspect of the invention, a scale for assessing at least one characteristic of an individual is provided wherein the scale comprises illustrations showing the mouth area of human subjects, the illustrations being organized in different categories representing levels of severity of the characteristic and wherein each level of severity is represented by at least two different human subjects. The characteristic is selected from the group consisting of lip fullness, severity of perioral lines at rest, severity of perioral lines at maximum contraction and oral commissures. In some embodiments the illustrations are photographs, though it is contemplated that the illustrations can be drawings, computer generated illustrations or other renderings.

In some embodiments, the level of severity of the characteristic is represented by photographs of at least two different human subjects. For example, each level of severity of the characteristic on the scale can be represented by photographs of two, three or more different human beings having the characteristic at the level being represented.

In some embodiments, the illustrations are organized in at least two rows and at least two columns wherein each row or each column shows a different level of severity of the characteristic.

Word-based descriptions adjacent each row may be provided wherein the descriptions correspond to the level of severity represented in the row or column to which it is adjacent.

In some embodiments at least one illustration, for example, all of the illustrations include a marking to guide a evaluator's attention to a specific area of said illustration.

In another aspect of the invention, a scale system for assessing at least one characteristic of the mouth area of an individual is provided, wherein the system comprises a lip fullness scale, a severity scale for perioral lines at rest, a severity scale for perioral lines at maximum contraction and an oral commissure severity scale.

In another aspect of the invention, a method for assessing at least one characteristic of an individual comprising providing a scale comprising illustrations showing the mouth area of human subjects, the illustrations being organized in different categories representing levels of severity of the characteristic and wherein each level of severity is represented by at least two different human subjects, and[ comparing the scale to a corresponding characteristic of an individual to obtain a rating of a level of severity of the corresponding characteristic of the individual.

### Scale Development

Approximately 300 untreated volunteers signed informed consent forms to undergo standardized digital photography of their lips. Images from both frontal and lateral perspectives, with the mouth relaxed and with the lips pursed, were obtained. Using verbal descriptions for each of the four grades for each scale, a board-certified dermatologist reviewed and scored 200 of the photos (Volunteers #001-#200) for Lip Fullness, Perioral Lines (at rest) and Oral Commissures. The verbal descriptions were presented as follows for each scale:

Lip Fullness (LFS) Scale
With Subject's Mouth at Rest:
(Assign the Grade that Corresponds to the
Subject's Worst Fullness Attribute)

| | | |
|---|---|---|
| A | None | Flat or nearly flat contour, minimal red lip show |
| B | Mild | Some red lip show; no lower lip pout |
| C | Moderate | Moderate red lip show with slight lower lip pout; may |
| | | have curves |
| D | Excellent | Significant red lip show and lower lip pout; may be very |
| | | curved |

Severity Scale For Perioral Lines At Rest (POL) Scale
With Subject's Mouth at Rest:
(Assign the Grade that Corresponds to the
Subject's Worst Perioral Lines Attribute)

| | | |
|---|---|---|
| D | None | No lines |
| C | Mild | Few, shallow lines |
| B | Moderate | Some, moderate lines |
| A | Severe | Many, deep lines or crevices |

Severity Scale For Oral Commissures (OCS) Scale
With Subject's Mouth Pursed:
(Assign the Grade that Corresponds to the Subject's
Worst Oral Commissures Attribute, Including the Marionette
Lines Only if Contiguous with the Mouth Corners)

| | | |
|---|---|---|
| D | None | No wrinkle or fold; slight upturned corners |
| C | Mild | Shallow, just perceptible wrinkle or crease; horizontal |
| | | or slightly downturned corners |
| B | Moderate | Moderately deep and/or long wrinkle or crease; |
| | | downturned corners |
| A | Severe | Very deep and/or long wrinkle or crease; frown at rest |

Based on this initial review and scoring as well as a technical review of the entire series of photos for standardized positioning, facial hair, scars or nevi in the lip area, facial expression, evenness of skin color, etc., a series of 95 photos were selected for review and scoring by two board-certified dermatologists and a board-certified ocular plastic surgeon.

The dermatologists reviewed the photos together and assigned one score for each scale for each photo. The dermatologists were encouraged to comment when they thought a specific subject would or would not be a good example for a particular scale and grade. The board-certified ocular plastic surgeon also provided a score. Photos for the LFS, POL, and OCS scales were selected from these 95 volunteers based on agreement in the two sets of scores and the reviewers' scale recommendations followed by the first dermatologist's selections from a final review of four photos per scale and grade.

Because the Perioral Lines at Maximum Contraction (POLM) scale is additionally intended for use in, without limitation, neurotoxin studies of the lips, it followed a different development path. In the development of the POLM scale, two board-certified dermatologists used verbal descriptions for each of the four grades to review and score all 300 volunteer photos. The verbal descriptions were presented as follows:

Severity Scale For Perioral Lines At Maximum Contraction (POLM)
With Subject's Mouth Pursed:
(Assign the Grade that Corresponds to the
Subject's Worst Vertical Lip Line Attribute)

| | | |
|---|---|---|
| D | None | No lines |
| C | Mild | Few, shallow lines |
| B | Moderate | Some, moderate lines |
| A | Severe | Many, deep lines or crevices |

Forty-nine sets of photos were selected from the 300 based on agreement between the two dermatologists' scores and distribution across all four grades. The images were then evaluated by five board-certified dermatologists for grading with verbal descriptions for each grade. Photos for the final POLM scale were selected based on agreement across the five reviewers followed by one of the first dermatologist's final review of about four photos per scale and grade.

Data from the POLM assessments were analyzed for computation of sample size for a scale validation study with multiple physicians. The kappa statistic was used for scale validation as it allows the measure of agreement beyond that expected by chance alone. Generally, kappa is calculated by estimating chance agreement and then comparing the observed agreement beyond chance with the maximum possible agreement beyond chance. In the present validation study, kappa estimates from the mKappa.sas macro were used. Due to their focus, these estimates are expected to be lower than weighted kappa estimates. Kappa scores in the range of 0.20 to 0.39 indicate fair agreement, 0.40 to 0.59 indicates moderate agreement, 0.60 to 0.79 indicates substantial agreement, and 0.80 to 1.00 indicates almost perfect agreement. See Landis, J R and Koch, G G, The measurement of observer agreement for categorical data, Biometrics 33:159-174 (1977) which is incorporated by reference in its entirety herein. Inter-rater agreement (i.e., between raters) was high among the five raters for the lowest and highest scores ((None (0) and Severe (3)), but was fair for the middle scores (Mild (1) and Moderate (2)) in this DVD photo review.

TABLE 1
POLM Inter-Rater Agreement Kappa Scores from Photo Review

| | | Standard | | |
|---|---|---|---|---|
| nscore2 | Kappa² | Error | z | Prob > Z |
| 0 | 0.75588 | 0.045175 | 16.7321 | <.0001 |
| 1 | 0.39302 | 0.045175 | 8.6998 | >.0001 |
| 2 | 0.23860 | 0.045175 | 5.2817 | <.0001 |
| 3 | 0.62677 | 0.045175 | 13.8742 | <.0001 |
| Overall | 0.49759 | 0.026175 | 19.0100 | <.0001 |

### Scale Validation

A group of eight physicians who were specialists in aesthetic dermatology/plastic surgery convened to validate all four scales. During pre-screening prior to the scale validation session, the Screening Investigator commented that some red lip will be visible even with the lowest level of Lip Fullness. That is, the Screening Investigator stated that the lowest grade for the LFS scale should be expressed as “Minimal,” rather than “None.” Additionally, the Screening Investigator recommended that the greatest level of Lip Fullness would be better expressed quantitatively as “Marked” rather than “Excellent.” Based on these recommendations, and at the outset of the session, the group agreed to change the verbal descriptions for the LFS scales and manually corrected these terms on the printed LFS scales before use. The remaining scales were not changed.

TABLE 2
Lip-Specific Effectiveness Scales

| | Perioral Lines | Perioral Lines | Oral |
|---|---|---|---|
| Lip Fullness | at Rest | at Maximum Contraction | Commissures |
| (LFS) | (POL) | (POLM) | (OCS) |
| Minimal | None | None | None |
| Mild | Mild | Mild | Mild |
| Moderate | Moderate | Moderate | Moderate |
| Marked | Severe | Severe | Severe |

After the eight physicians underwent training on the scale validation protocol and in the use of the scale, they were seated in a large circle in a well-lit meeting room facing away from each other, i.e., with their backs toward the center of the circle. Each sat at a small table, which was fitted with a large envelope that hung from the side of the table and an 8½ inch vertical partition taped to the tabletop. On the rater's side of the partition were four stacks of cards, corresponding to the four grades on the photographic scales.

A group of 55 pre-screened volunteers formed a queue based on an a priori random order for each scale to be validated. Each subject received a sheet of pre-printed labels with his/her subject number, round number (1 or 2), and the scale name. As the subjects entered the meeting room, a doorkeeper instructed them as to which scale and which set of labels were to be used during the upcoming evaluation. They moved from rater to rater in sequence, sitting across the table from the rater for each evaluation and providing the appropriate pre-printed label to the rater. Behind the partition the rater selected a card to correspond to the subject's score for the specific scale, applied the subject's label to the card, and dropped the card into the large envelope. This sequence was performed once for all subjects, all raters, and all four scales in the morning (Round 1), and it was repeated in the afternoon (Round 2).

### Physician Inter- and Intra-Rater Agreement

Overall, intra-rater agreement (Round 1 compared to Round 2 scores) was high for all four scales. For example, the standard deviation for intra-rater agreement across the eight raters using the Lip Fullness scale was 0.044 with a mean weighted Kappa coefficient of 0.799 (95% Cl 0.762-0.836).

TABLE 3
Live Assessments: Weighted Kappa Scores for Intra-Rater Agreement

| | | Perioral Lines | Perioral Lines | Oral |
|---|---|---|---|---|
| | Lip Fullness | at Rest | at Maximum Contraction | Commissures |
| Statistic | (LFS) | (POL) | (POLM) | (OCS) |
| Mean (±SD) | 0.799 (±0.0440) | 0.725 (±0.088) | 0.826 (±0.050) | 0.789 (±0.039) |
| 95% CI | 0.762-0.836 | 0.652-0.799 | 0.784-0.867) | 0.756-0.821 |

Inter-rater agreement was measured by intra-class correlation (ICC) a measure of the proportion of reliable variance. Intra-class correlation for the LFS scale was 0.814 and 0.787 at Round 1 and Round 2, respectively. By grade 2 within each of the four scales the Kappa estimates indicate that inter-rater agreement was generally moderate to substantial.

TABLE 4
Intra-Class Correlations (ICC) for Inter-Rater Agreement

| | | Perioral Lines | Perioral Lines | Oral |
|---|---|---|---|---|
| | Lip Fullness | at Rest | at Maximum Contraction | Commissures |
| Statistic | (LFS) | (POL) | (POLM) | (OCS) |
| Round 1 | | | | |
| SF Single | 0.814 | 0.809 | 0.853 | 0.817 |
| SF Mean K | 0.976 | 0.853 | 0.981 | 0.977 |
| Round 2 | | | | |
| SF Single | 0.787 | 0.818 | 0.873 | 0.818 |
| SF Mean K | 0.973 | 0.980 | 0.980 | 0.977 |

### Subject Inter-Rater and Intra-Rater Agreement

Subjects provided two series of self-assessments using the same four lip-specific photographic scales as the physician raters. Intra-rater agreement between Round 1 and Round 2 was substantial for all four scales. Inter-rater agreement with the physician raters was substantial for all four scales for both Round 1 and Round 2 scores.

TABLE 5
Inter-Rater Agreement: Subject Self-Assessments vs. Physician Raters

| | | Perioral Lines | Perioral Lines | Oral |
|---|---|---|---|---|
| | Lip Fullness | at Rest | at Maximum Contraction | Commissures |
| | (LFS) | (POL) | (POLM) | (OCS) |
| Statistic | n = 55 | n = 54 | n = 55 | n = 55 |
| Subject Mean (±SD) | 2.96 (0.838) | 1.76 (0.816) | 2.60 (0.974) | 2.27 (1.008) |
| Physician Mean (±SD) | 2.88 (0.874) | 1.98 (0.852) | 2.69 (1.001) | 2.32 (0.947) |
| Round 1 ICC | 0.7995 | 0.6820 | 0.7606 | 0.8057 |
| Subject Mean (±SD) | 2.93 (0.949) | 1.89 (0.883) | 2.61 (0.940) | 2.22 (01.076) |
| Physician Mean (±SD) | 2.88 (0.835) | 2.09 (0.954) | 2.74 (0.967) | 2.34 (0.972) |
| Round 2 ICC | 0.7546 | 0.7787 | 0.7370 | 0.7682 |

The described results demonstrate that each of the four lip-specific photographic scales of the invention has a Kappa score of at least about 0.700. The scales have been validated to be reliable (reproducible) and consistent for physician classification of aesthetic features of the lips and mouth area of live subjects. Inter-rater agreement by grade within the scales improved with live scoring of volunteers compared with the scoring of digital images.

Subject self-assessments are also reliable and consistent and comparable to physician assessments using the four lip-specific photographic scales for the classification of aesthetic features of the lips and mouth area of live subjects.

Based on these results, the present invention provides scales and methods that provide reliable scales, for example, scales having a Kappa score of at least about 0.700 for intra-rater and inter-rater reliability, to effectively and consistently rate lip fullness and the severity of perioral lines or oral commissures.

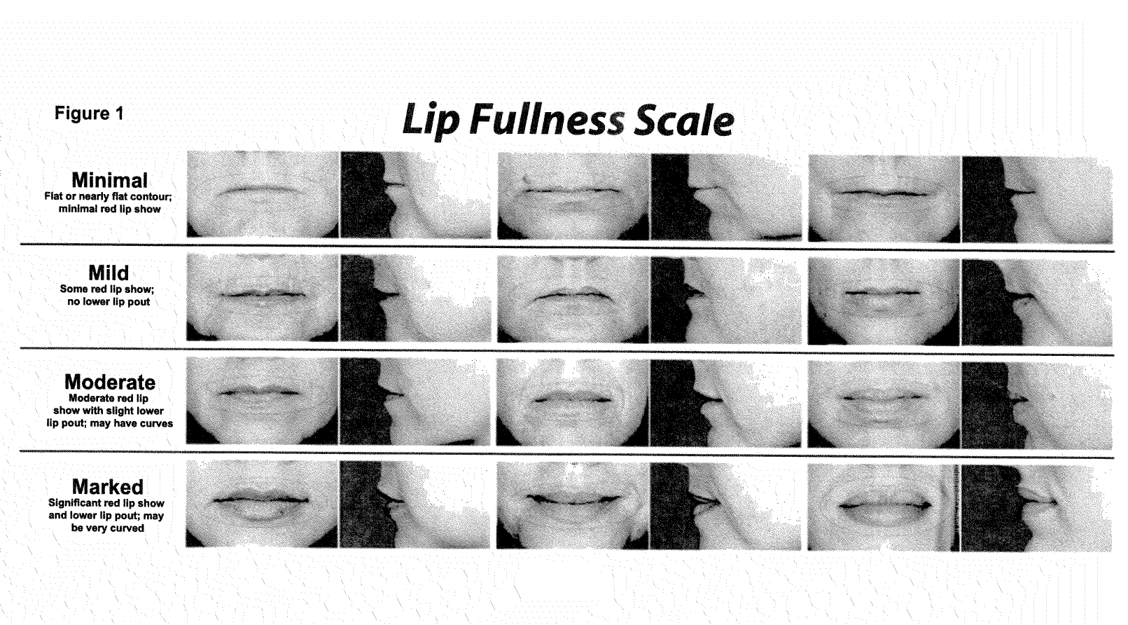

FIG. 1 depicts a LFS scale. As can be seen, in this depicted embodiment, four rows of photographs are provided with each row corresponding to a degree or severity of lip fullness. The degrees are characterized as Minimal, Mild, Moderate and Marked. Each of these word-based descriptions for the degree or severity of the rating is followed by explanatory words describing a feature commonly found in the corresponding row of photographs. This depicted LFS scale provides a frontal view and a side view of sample lips that would correspond to their assigned severity category. In this depicted embodiment, the frontal view contains markings to guide the rater's attention to the area of interest. As will be understood by one of ordinary skill in the art, the markings described herein can be any appropriate marking including, without limitation, lines or shading.

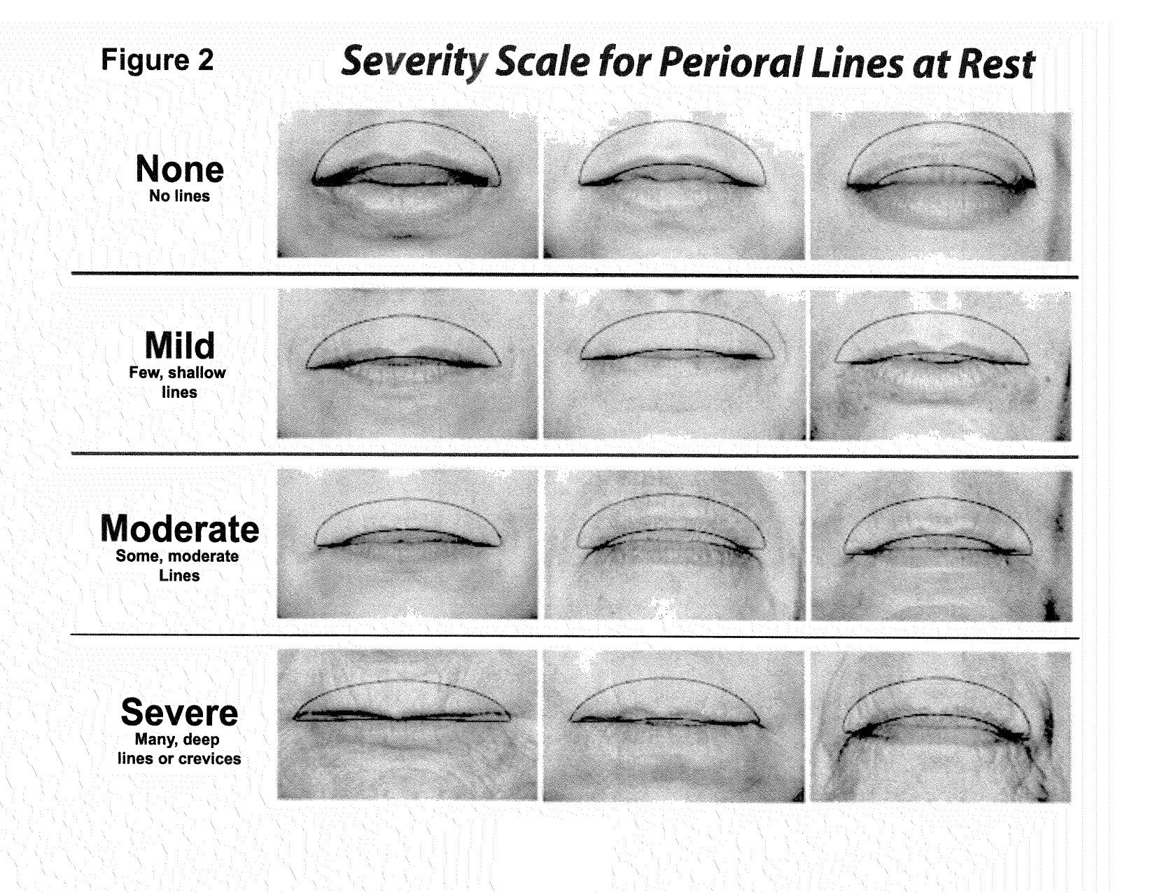

FIG. 2 depicts a POL scale. As can be seen, in this depicted embodiment, four rows of photographs are provided with each row corresponding to a degree or severity of perioral lines at rest. Markings are provided on the photograph to guide a rater's attention to the appropriate area around the mouth for the particular rating. The degrees are characterized as None, Mild, Moderate and Severe. Each of these word-based descriptions for the degree or severity of the rating is followed by explanatory words describing a feature commonly found in the corresponding row of photographs. This depicted POL scale depicts a frontal view of sample mouth and lip areas that would correspond to their assigned severity category.

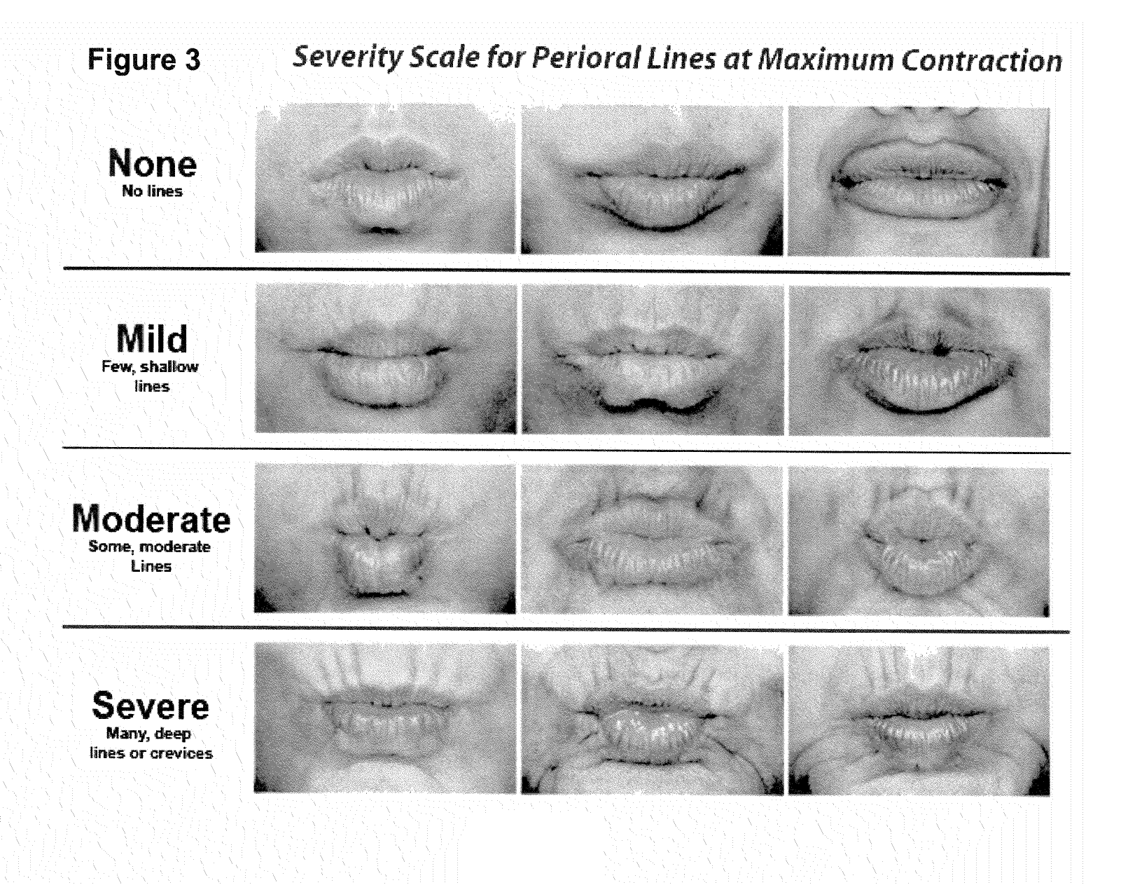

FIG. 3 depicts a POLM scale. As can be seen, in this depicted embodiment, four rows of photographs are provided with each row corresponding to a degree or severity of perioral lines at maximum contraction. The degrees are characterized as None, Mild, Moderate and Severe. Each of these word-based descriptions for the degree or severity of the rating is followed by explanatory words describing a feature commonly found in the corresponding row of photographs. This depicted POLM scale depicts a frontal view of sample mouth and lip areas that would correspond to their assigned severity category. Markings can be provided to guide the rater's attention to the area of interest.

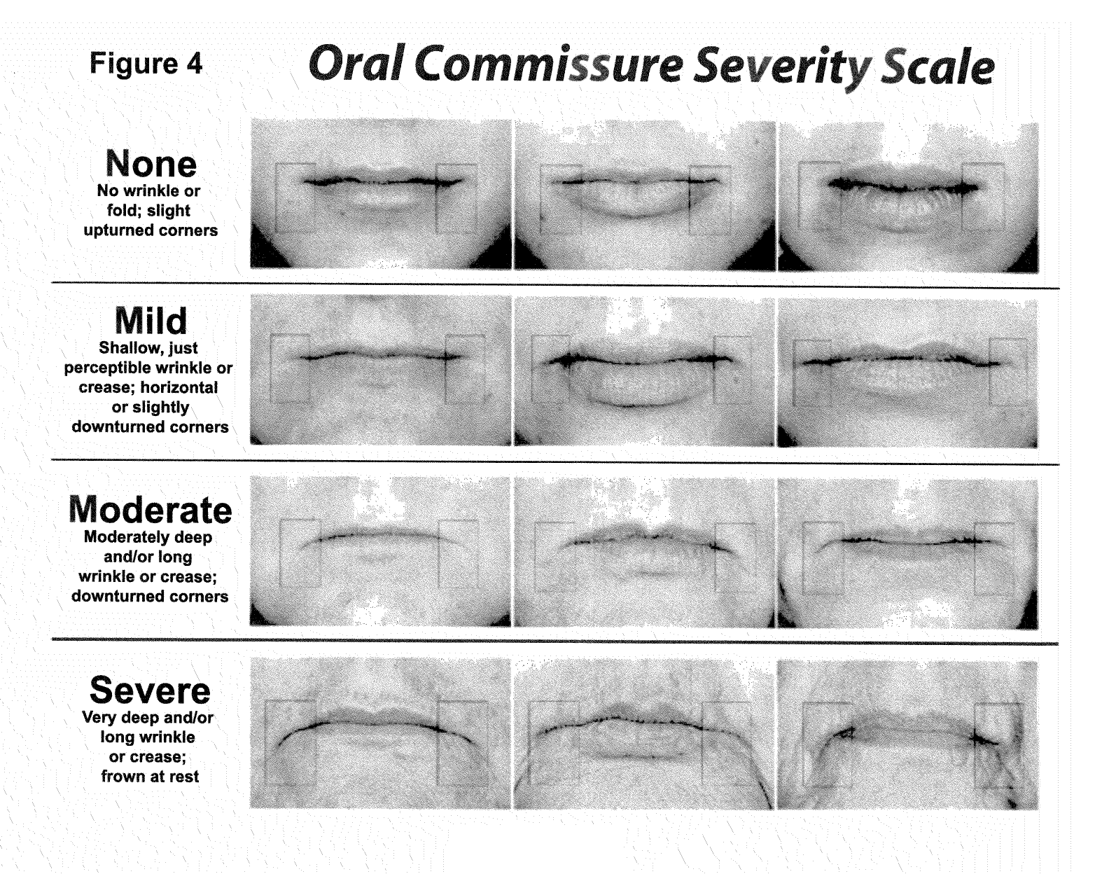

FIG. 4 depicts an OCS scale. As can be seen, in this depicted embodiment, four rows of photographs are provided with each row corresponding to a degree or severity of oral commissures. Markings are provided on the photograph to guide a rater's attention to the appropriate area around the mouth for the particular rating. The degrees are characterized as None, Mild, Moderate and Severe. Each of these word-based descriptions for the degree or severity of the rating is followed by explanatory words describing a feature commonly found in the corresponding row of photographs. This depicted OCS scale depicts a frontal view of sample mouth and lip areas that would correspond to their assigned severity category.

These scales can be used to reliably and consistently to document the effectiveness of treatments during regular clinical therapy and during clinical trials testing treatment efficacy for therapies or treatments addressing lip fullness, perioral lines or oral commissures.

### Example

### Use of Scales of the Invention in a Clinical Study to Assess Safety and Effectiveness of an HA Filler in the Use of Cosmetic Lip Enhancement

A clinical study was designed to collect data on the safety and effectiveness of a dermal filler for lip enhancement. The present scales and methods were used in the study.

In this Example, the dermal filler studied was JUVÉDERM™ Ultra Injectable Gel.

JUVÉDERM™ Ultra Injectable Gel is a sterile, biodegradable, non-pyrogenic, viscoelastic, clear, colorless, homogenized gel implant (dermal filler). It comprises a crosslinked hyaluronic acid (HA) formulated to a concentration of 24 mg/mL, suspended in a physiological buffer. The HA in JUVÉDERM™ Gel is produced by Streptococcus equi bacteria and has a molecular weight of approximately 2.5 million Daltons. The HA is mixed with phosphate buffer and cross-linked by adding a minimum amount of BDDE (1,4-butanediol diglycidyl ether) to form a 3-dimensional HA gel.

### Overall Study Design and Plan

Eight research centers participated in an open label feasibility study under an Investigational Device Exemption (IDE). The study treatment sites included one or more areas of the cutaneous and mucosal lips and around the mouth (e.g., vermilion, vermilion borders, Cupid's bow and philtral columns, and oral commissures).

A Treating Investigator (TI) at each center determined an appropriate volume of material to inject into the treatment site of each subject, with an allowed a maximum total volume of 4 syringes (3.2 mL) per study subject. The TI determined, using ordinary skill in the art, an appropriate volume to administer in the perioral treatment areas (vermilion, vermilion borders, Cupid's bow and philtral columns, and oral commissures) depending on the subject's treatment goals.

At each visit, standardized digital 3D facial images of the subject were obtained. The TIs and Evaluating Investigators (EIs) and the subject assessed the subject's lips for fullness, perioral lines and oral commissures using the respective validated 4-point scales and determine whether the subject's lip fullness treatment goal has been met.

The TIs and EIs also assessed other aesthetic and functional features of the lips and mouth area. The subject assessed the look and feel of his/her lips and mouth and his/her willingness to undergo treatment again.

EIs were blinded to the volume and location of treatments and previous assessment scores for all study subjects.

Subjects were informed that they are not allowed to begin use of any new over-the-counter or prescription, oral or topical, anti-wrinkle products for the lips or around the mouth within 4 weeks prior to enrollment or at any time during the study.

At baseline and throughout the study, the investigator recorded concomitant medications, therapies, and treatments.

A primary effectiveness measure was based on a combination of the Evaluating Investigator's assessment of Lip Fullness Score (LFS) using the 4-point photographic scale and the subject's assessment of whether the treatment goal was achieved at Week 12.

A primary effectiveness variable is the derived variable of “responder.” A “responder” is considered to be any subject whose LFS rating was improved by 1 point or more from pre- to post-treatment and who had achieved his/her LFS treatment goal.

The study device was determined to be clinically effective if at least 40% of subjects demonstrated clinically significant improvement (increase ≧1-point on a 4-point scale) based on the Evaluating Investigator's assessment of the subjects' Lip Fullness compared with their pre-treatment Lip Fullness assessment scores and had achieved their LFS treatment goals based on the subjects' assessments at 12 weeks after last treatment.

Secondary effectiveness measurements included subjects' and investigators' assessments of whether the lip fullness goals have been achieved, subjects' self-assessments and both investigators' assessments of lip fullness, perioral lines, and oral commissures; investigators' assessments of other aesthetic features of subjects' lips and mouth area and subjects' assessments of the look and feel of their lips and mouth area (both using 11-point scales). Assessments were made at baseline and each follow-up visit.

Exploratory analyses included a summary of lip measurements for vertical red lip height, anterior lip projection, mathematical estimate of lip volume, and mathematical estimate of lip volume change obtained from standardized 3D digital images taken by trained staff members at baseline and each follow-up visit. Computerized calculations of these measurements were performed by a photographic processing vendor and the volume estimates were computed using an algorithm and verified by an independent mathematician.

The TIs and EIs examined inside and around the subject's lips and mouth, at rest and in animation (smiling, frowning, etc), for lip function, texture, discoloration, firmness, lumpiness, or mucoceles. Findings from this examination and subjects' self-assessments for function and sensation of the lips and mouth were recorded at baseline and each follow-up visit.

Intra- and inter-rater reliability was established for the three scales utilized by investigators and subjects for assessments of the primary measure, lip fullness, and the secondary measures, severity of perioral lines and severity of oral commissures.

All TIs and EIs were trained on the use of the scales prior to subject enrollment. The verbiage for the scales is provided in Tables 2.1, 2.2 and 2.3, and the full scales with photographic guides are provided such as shown in FIGS. 1-4.

TABLE 2.1
Lip Fullness Scale

| Score | Description |
|---|---|
| Minimal | Flat or nearly flat contour, minimal red lip show |
| Mild | Some red lip show; no lower lip pout |
| Moderate | Moderate red lip show with slight lower lip pout; |
| | may have curves |
| Marked | Significant red lip show and lower lip pout; |
| | may be very curved |

TABLE 2.2
Perioral Lines Severity Scale

| Score | Description |
|---|---|
| None | No lines |
| Mild | Few, shallow lines |
| Moderate | Some, moderate lines |
| Severe | Many, deep lines or crevices |

TABLE 2.3
Oral Commissure Severity Scale

| Score | Description |
|---|---|
| None | No wrinkle or fold; slight upturned corners |
| Mild | Shallow, just perceptible wrinkle or crease; |
| | horizontal or slightly downturned corners |
| Moderate | Moderately deep and/or long wrinkle or |
| | crease; downturned corners |
| Severe | Very deep and/or long wrinkle or crease; |
| | frown at rest |

Objective linear measurements of vertical red lip heights and anterior lip projection as well as mathematical estimates of lip volume and volume change for all subjects was recorded from 3D digital images that were obtained in a standardized fashion before and after treatment.

Subject disposition is presented in Table 2.4. Fifty-five subjects were enrolled in the study. Fifty subjects were treated. Five subjects did not undergo treatment due to ineligibility (n=4) or withdrawal from study (n=1).

TABLE 2.4
Disposition of Subjects

| | n | % |
|---|---|---|
| Enrolled Subjects | 55 | 100% |
| Underwent study device treatment (ITT population) | 50 | 91% |
| Completed the study as per protocol | N/A | |
| Did not complete the study as per protocol | N/A | |
| Did not undergo study device treatment | 5 | 9% |
| Ineligible/Excluded | 4 | 7% |
| Withdrew from study | 1 | 2% |

Most of the subjects are women (96%) of Caucasian descent (88%) with Fitzpatrick skin type II or III (80%). Median age at study entry is 47 years. Table 2.5 presents subject demographics.

TABLE 2.5
Subject Demographics

| | n | % |
|---|---|---|
| Gender | | |
| Female | 48 | 96% |
| Male | 2 | 4% |
| Age (years) | | |

| | |
|---|---|
| Mean | 47.6 |
| Median | 47.0 |
| Range | 24-68 |

| | | |
|---|---|---|
| Ethnicity (Number %) | | |
| Caucasian | 44 | 88% |
| African American | 1 | 2% |
| Latino/Latina | 5 | 10% |
| Asian | 0 | 0% |
| Other | 0 | 0% |
| Fitzpatrick Skin Type | | |
| I | 1 | 2% |
| II | 22 | 44% |
| III | 18 | 36% |
| IV | 7 | 14% |
| V | 1 | 2% |
| VI | 1 | 2% |

(N = 50 Subjects)

Anesthesia was administered in 92% of initial treatments and 78% of touch-up treatments. Nerve block, local, and topical anesthesia was used in fairly even proportions in preparation for treatment, often in combination with each other.

Of 50 subjects who received an initial treatment, 18 (36%) also received a touch-up treatment. The median number of syringes used was 2 for initial treatments and 1 for touch-up treatments.

Table 2.6 details the injection sites for initial treatment and touch-up treatments. All subjects (100%) received injections in the upper lip vermilion border and almost all (92%) in the lower lip vermilion border as well. The vermilion (mucosa) of both the upper and lower lips was also often treated (in 48 and 46 subjects, respectively). Cupid's bow was treated in 40 subjects (80%), and philtral columns were treated in 16 subjects (32%). Oral commissures were also frequently treated (43 subjects, 86%).

TABLE 2.6
Injection Site

| | Initial Treatment | | Touch-up Treatment | |
|---|---|---|---|---|
| | (N = 50) | | (N = 18) | |
| Site* | n | % | n | % |
| Upper lip | 50 | 100% | 12 | 67% |
| Vermilion Border | 50 | 100% | 8 | 44% |
| Vermilion (Mucosa) | 48 | 96% | 11 | 61% |
| Cupid's Bow | 40 | 80% | 4 | 22% |
| Philtral Columns | 16 | 32% | 0 | 0% |
| Lower Lip | 50 | 100% | 10 | 56% |
| Vermilion Border | 46 | 92% | 7 | 39% |
| Vermilion (Mucosa) | 46 | 92% | 8 | 44% |
| Oral Commissures | 43 | 86% | 12 | 67% |

A variety of injection planes were utilized in combination to obtain optimal results. 43 subjects were treated using sub-dermal injections. Intra-dermal injections were used in 34 subjects and intra-muscular are used in 25 subjects. Sub-dermal was the most frequent injection plane for the vermilion border, vermilion and Cupid's bow whereas intra-dermal was the most common for philtral columns and oral commissures. Most of the intra-muscular injections (23 of 34) were in the vermilion.

Multiple injection techniques were also used in combination with each other. Retro-grade tunneling was used most frequently during initial treatments (80% of subjects), followed by ante-grade tunneling (48%), serial puncture (40%), cross-hatching (34%), and fanning (18%). Cross-hatching and fanning were used exclusively for oral commissures. For touch-up treatments, retro-grade tunneling was most frequently used.

The median volume injected per subject was 1.6 mL (2 syringes) at the initial treatment (range, 0.65 to 3.2 mL) and 0.6 mL at the touch-up treatment (range, 0.1 to 1.7 mL). The volume was fairly evenly distributed among the three locations, with a median of 0.7 mL for the upper lip, 0.5 mL for the lower lip, and 0.5 mL for the oral commissures at initial treatment.

### Lip Fullness

Prior to treatment, the subject and TI developed a treatment goal for lip fullness based on a lip fullness scale, such the lip fullness scale shown in FIG. 1. The EI, TI, and subject provide assessments of lip fullness at baseline, Week 2, Week 6, and Week 12.

At baseline, the EIs rate lip fullness as minimal for 30% of subjects, mild for 64%, and moderate for 6%. After treatment at Week 2, lip fullness had improved such that no subjects were rated as minimal, 14% were mild, 56% were moderate, and 30% were marked. At the Week 12 visit, no subjects had regressed to a fullness of minimal, and 31% are mild, 49% moderate, and 20% marked.

At Week 2, 88% of subjects were demonstrating improvement of at least 1 point in fullness score assessed by the EIs. By Week 12, improvement of at least 1 point was still high at 80% of subjects. Subjects' self-assessments followed patterns similar to those of the EIs.

The overwhelming majority of subjects (90%-96%) were determined by both the EI and subject to have met the pre-treatment lip fullness goal at each timepoint. A significant proportion of subjects (p<0.0001) had achieved their goals and also showed improvement of ≧1 point based on the EI's assessment of lip fullness at the Week 12 timepoint.

TABLE 2.7
Lip Fullness Clinical Effectiveness at Week 12

| Evaluating Investigator's | Subject's Self Assessment | | | | |
|---|---|---|---|---|---|
| Assessment of Change in | Goal Achieved | | | Goal Not Achieved | |
| Lip Fullness Score | n | % | p-value* | n | % |
| Improved ≧1 pt | 35 | 71% | <.0001 | 4 | 8% |
| No Change | 9 | 18% | | 1 | 2% |
| Worse | 0 | 0% | | 0 | 0% |

In total, 71% of subjects were classified as responders compared with the 40% needed to demonstrate clinical effectiveness.

Perioral lines were assessed on a scale of none, mild, moderate, and severe. Achievement goals were not predetermined in regard to perioral lines. The EI, Pl, and subject provided assessments of perioral line severity at baseline, Week 2, Week 6, and Week 12.

At baseline, EIs determined that 20% of subjects had no perioral lines, 40% had mild lines, 30% moderate, and 10% severe. By the Week 12 visit, 39% of subjects had no perioral lines, 45% mild, 14% moderate and 2% severe. Overall, the EIs noted that perioral line severity improved at least 1 point from baseline to Week 12 for 51% of subjects. Subjects' self-assessments were similar to those of the EIs, with 63% noting improvement of at least 1 point from baseline to Week 12 (Table 2.8).

TABLE 2.8
Change in Perioral Line Severity Pre- vs. Post-Treatment

| | | | Change in Severity | | | | | | | |
|---|---|---|---|---|---|---|---|---|---|---|
| | | | Improved >1 pt | | Improved 1 pt | | No Change | | Worse | |
| | Week | N | n | % | n | % | n | % | n | % |
| Evaluating | Week 2 | 50 | 0 | 0% | 22 | 44% | 28 | 56% | 0 | 0% |
| Investigator's | Week 6 | 49 | 3 | 6% | 22 | 45% | 22 | 45% | 2 | 4% |
| Assessment | Week 12 | 49 | 0 | 0% | 25 | 51% | 23 | 47% | 1 | 2% |
| Treating | Week 2 | 50 | 8 | 16% | 26 | 52% | 15 | 30% | 1 | 2% |
| Investigator's | Week 6 | 49 | 7 | 14% | 28 | 57% | 14 | 29% | 0 | 0% |
| Assessment | Week 12 | 49 | 4 | 8% | 29 | 59% | 16 | 33% | 0 | 0% |
| Subject's Self | Week 2 | 50 | 7 | 14% | 32 | 64% | 10 | 20% | 1 | 2% |
| Assessment | Week 6 | 49 | 6 | 12% | 28 | 57% | 11 | 22% | 4 | 8% |
| | Week 12 | 49 | 5 | 10% | 26 | 53% | 16 | 33% | 2 | 4% |

### Oral Commissures

Oral commissures were evaluated on a 4-point scale, such as the scale shown in FIG. 4. Oral commissures were treated in 43 of the 50 subjects (86%).

The EIs determined that 6 subjects (14%) had severe oral commissures at baseline and 2 (5%) at Week 12; the frequency of moderate oral commissures decreased from 25 subjects (58%) at baseline to 6 subjects (14%) at Week 12. Subjects' self-assessments showed similar trends.

An improvement of at least 1 point in oral commissure severity from baseline to Week 12 was noted for 64% of subjects per the EIs' assessments. Similar values were obtained from the subjects' self-assessments.

### Aesthetic Features

EIs and TIs physically examined various aesthetic features of the subjects pre- and post-treatment. A 10-point scale was used for the assessments, where 0 is Not At All, 5 is Somewhat, and 10 is Very Much. In all cases, there was a substantial increase in the number of subjects who were scored in the 7-10 (“Very Much”) range of the scale from baseline to Week 12 (Table 2.9).

TABLE 2.9
Evaluating Investigators Assessment of Aesthetic Features

| | Week | Score* | |
|---|---|---|---|
| | Baseline N = 50 | 7-10 (Very Much) | |
| Aesthetic Feature | Week 12 N = 49 | n | % |
| Accepted Vertical Proportions | Baseline | 30 | 60% |
| for Lower Face | Week 12 | 45 | 92% |
| Upper & Lower Lips Conform to | Baseline | 21 | 42% |
| Accepted Relative Proportion | Week 12 | 43 | 88% |
| Lip Shape and Size are | Baseline | 23 | 46% |
| Proportional Relative to Face | Week 12 | 42 | 86% |
| Vermilion Borders are Sharp | Baseline | 19 | 38% |
| and Well-Defined | Week 12 | 41 | 84% |
| Peaks of Cupid's Bow are | Baseline | 20 | 40% |
| Distinct, and Well-Defined | Week 12 | 39 | 80% |
| Philtrum | | | |
| Proportionate Upper and Lower | Baseline | 24 | 48% |
| Central Lip Fullness | Week 12 | 43 | 88% |
| Oral Commissure Fullness to | Baseline | 16 | 32% |
| Support Lateral Aspect of Lip | Week 12 | 44 | 90% |
| Symmetrical Right and Left | | | |
| Sides of Lips and Mouth | | | |
| In Repose | Baseline | 34 | 68% |
| | Week 12 | 45 | 92% |
| In Animation | Baseline | 35 | 70% |
| | Week 12 | 46 | 94% |

*0 = Not At All, 5 = Somewhat, and 10 = Very Much

According to EIs, overall satisfaction with aesthetic features of subject's lips and mouth area had increased from 42% very satisfied at baseline to 90% very satisfied at Week 12 for subjects in both repose and animation.

Multiple assessments were made by subjects pre- and post-treatment regarding the look and feel of the lips and mouth using a 10-point scale where 0 is Not At All, 5 is Somewhat, and 10 is Very Much. Table 2.10 shows that in all cases, there was a dramatic improvement in aesthetic features from baseline to Week 12.

TABLE 2.10
Subject Assessment of Aesthetic Features

| | Week | Score* | |
|---|---|---|---|
| | Baseline N = 50 | 0-3 (Not at All) | |
| Aesthetic Feature | Week 12 N = 49 | n | % |
| Lips and Mouth Detract from | Baseline | 7 | 14% |
| the Facial Appearance | Week 12 | 43 | 88% |
| Bothered by Lips and Mouth | Baseline | 10 | 20% |
| | Week 12 | 42 | 86% |
| Lips and Mouth Make Subject | Baseline | 6 | 12% |
| Look Older | Week 12 | 40 | 82% |
| Lips and Mouth Make Subject | Baseline | 11 | 22% |
| Look Sad | Week 12 | 42 | 86% |
| Lips and Mouth Make Subject | Baseline | 13 | 26% |
| Look Angry | Week 12 | 42 | 86% |
| Lip and Mouth Make Subject | Baseline | 10 | 20% |
| Look Unattractive | Week 12 | 43 | 88% |
| Lipstick ‘Bleeds’ into the | Baseline | 15 | 31% |
| Lines Above/Below the Edges | Week 12 | 42 | 86% |
| of Lips | | | |
| Lips and Mouth Feel Hard | Baseline | 27 | 54% |
| | Week 12 | 45 | 92% |
| Lips and Mouth Feel Lumpy/ | Baseline | 25 | 50% |
| Grainy | Week 12 | 44 | 90% |
| Lips and Mouth Feel Unnatural | Baseline | 27 | 55% |
| | Week 12 | 44 | 90% |
| Lips and Mouth Look Uneven | Baseline | 17 | 34% |
| | Week 12 | 45 | 92% |
| Lips and Mouth Look Unnatural | Baseline | 27 | 55% |
| | Week 12 | 45 | 92% |

*0 = Not At All, 5 = Somewhat, and 10 = Very Much

Overall, subjects were satisfied with the effects of the study treatment at each follow-up visit (Table 2.11).

TABLE 2.11
Subject's Overall Satisfaction with the Effect of Study Treatment

| | | Score* | | | | | |
|---|---|---|---|---|---|---|---|
| | | 0-3 | | | | 7-10 | |
| | | Very Satisfied | | 4-6 | | Very Dissatisfied | |
| Week | N | n | % | n | % | n | % |
| Week 2 | 49 | 45 | 92% | 3 | 6% | 1 | 2% |
| Week 6 | 49 | 43 | 88% | 2 | 4% | 4 | 8% |
| Week 12 | 49 | 40 | 82% | 6 | 12% | 3 | 6% |

*Score ranges from 0 to 10 where 0 = Very Satisfied and 10 = Very Dissatisfied

### 3D Digital Images

Exploratory analyses of effectiveness of lip measurements obtained from 3D digital images include vertical red lip heights, anterior lip projection, lip volume and volume change estimates. Table 2.12 presents the measurement description, an example, the mean and comparison of mean to baseline for each follow-up visit.

All of the measurements of lip height show increases over baseline at each timepoint. The largest increase is observed in the lower red lip height for frontal view (22%-25% increase from baseline).

From a lateral perspective, lip projection is measured in a forward (horizontal) dimension. The lip projection measurement is the perpendicular distance from the most forward aspect of the lip to a reference line drawn from the tip of the nose to the chin, shown in Table 2.12. As lip fullness increases, the distance to the reference line decreases resulting in a decrease in the lip projection measurement compared to baseline.

There was a positive change in lip volume since baseline, with the change greatest at Week 2 (0.90 cc) and sustained through Week 12 (0.75 cc). Overall, the mean surface area of the lips had increased from 6.83 cm² at baseline to 8.77 cm² at Week 2 (33% increase) and 8.28 cm² at Week 12 (25% increase).

### Effectiveness Conclusions

Through use of at least one or more of the scales and methods of the present invention, the tested dermal filler, JUVÉDERM™ Ultra, was demonstrated to meet pre-established criteria for clinical effectiveness in lip enhancement, with 71% of subjects classified as responders at Week 12.

Except for the categorical numerical values assigned to the grades of the described scales, and unless otherwise indicated, all numbers expressing quantities or properties and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A scale for assessing at least one characteristic of an individual, the scale comprising:
illustrations showing the mouth area of human subjects, the illustrations being organized in different categories representing levels of severity of the characteristic and wherein each level of severity is represented by at least two different human subjects; and
the characteristic being selected from the group consisting of lip fullness, severity of perioral lines at rest, severity of perioral lines at maximum contraction and oral commissures.

2. The scale of claim 1 wherein the illustrations are photographs.

3. The scale of claim 2 wherein level of severity of the characteristic is represented by photographs of at least two different human subjects.

4. The scale of claim 1 wherein the illustrations are organized in at least two rows and at least two columns.

5. The scale of claim 4 wherein each row shows a different level of severity of the characteristic.

6. The scale of claim 6 wherein the illustrations are photographs and each row includes photographs taken from at least two different human subjects.

7. The scale of claim 1 having a Kappa score of at least about 0.700 for intra-rater and inter-rater reliability.

8. The scale of claim 1 wherein said characteristic is lip fullness.

9. The scale of claim 1 wherein said characteristic is severity of perioral lines.

10. The scale of claim 1 wherein said characteristic is severity of perioral lines at rest.

11. The scale of claim 1 wherein said characteristic is severity of perioral lines at maximum contraction.

12. The scale of claim 1 wherein said characteristic is oral commissures.

13. The scale according to claim 4 further comprising word-based descriptions adjacent each row, the descriptions corresponding to the level of severity represented in the row to which it is adjacent.

14. The scale according to claim 1 wherein at least one illustration comprises a marking to guide a evaluator's attention to an area of said illustration.

15. A scale system for assessing at least one characteristic of the mouth area of an individual, the system comprising:
a lip fullness scale comprising illustrations showing the mouth area of human subjects, the illustrations being organized in different categories representing levels of lip fullness;
a severity scale for perioral lines at rest comprising illustrations showing the mouth area of human subjects, the illustrations being organized in different categories representing levels of severity of perioral lines at rest; and
an oral commissure severity scale comprising illustrations showing the mouth area of human subjects, the illustrations being organized in different categories representing levels of severity of oral commissure.

16. The system of claim 15 further comprising a severity scale for perioral lines at maximum contracture comprising illustrations showing the mouth area of human subjects, the illustrations being organized in different categories representing levels of severity of perioral lines at maximum contracture.

17. The system of claim 15 wherein the illustrations are photographs.

18. The system of claim 15 wherein each level of lip fullness is illustrated by photographs of at least two different human subjects.

19. The system of claim 15 wherein each level of severity of perioral lines is illustrated by photographs of at least two different human subjects.

20. The system of claim 15 wherein each level of severity of perioral lines is illustrated by photographs of at least three different human subjects.

21. The system of claim 15 wherein each level of severity of oral commissure is illustrated by photographs of at least two different human subjects.

22. The system of claim 15 wherein each level of severity of oral commissure is illustrated by photographs of at least three different human subjects.

23. A method for assessing at least one characteristic of an individual comprising:
providing a scale comprising illustrations showing the mouth area of human subjects, the illustrations being organized in different categories representing levels of severity of the characteristic and wherein each level of severity is represented by at least two different human subjects, the characteristic being selected from the group consisting of lip fullness, severity of perioral lines, severity of perioral lines at maximum contraction and oral commissures; and
comparing the scale to a corresponding characteristic of an individual to obtain a rating of a level of severity of the corresponding characteristic of the individual.

24. The method of claim 23 wherein level of severity of the characteristic is represented by photographs of at least two different human subjects.
